(19) United States
(12) Patent Application Publication
(10) Pub. No.: US 2022/0152422 A1
(43) Pub. Date: May 19, 2022

(54) MARKING METHOD FOR MARKING RADIATION THERAPY POSITIONING NOTE

(71) Applicant: CENTRON BIOCHEMISTRY TECHNOLOGY CO., LTD., Tainan (TW)

(72) Inventor: Chun-Pi LIN, Tainan (TW)

(73) Assignee: CENTRON BIOCHEMISTRY TECHNOLOGY CO., LTD., Tainan (TW)

(21) Appl. No.: 17/098,083

(22) Filed: Nov. 13, 2020

(51) Int. Cl. A61N 5/10; A61F 13/00; A61F 13/02

(52) U.S. Cl. CPC A61N 5/1049; A61B 2090/3937; A61F 13/0259; A61F 13/00059

(57) ABSTRACT

Disclosed is a marking method for marking a radiation therapy positioning note, comprising in sequence: a preparing step of providing a radiation therapy positioning sticker, wherein the radiation therapy positioning sticker includes a pattern layer and a release layer disposed on an outer surface of the pattern layer, and the radiation therapy positioning note is formed on the outer surface of the pattern layer; a transferring step of attaching the radiation therapy positioning sticker onto a predetermined note position of a radiation therapy target object in a manner that an inner surface of the pattern layer faces forward the predetermined note position and covers the predetermined note position; and a removing step of peeling off the release layer from the outer surface of the pattern layer to outwardly expose the radiation therapy positioning note, which is formed on the outer surface of the pattern layer covering the predetermined note position.

### FIELD OF THE INVENTION

The present invention relates to a radiation therapy positioning note, and more particularly relates to a marking method for marking radiation therapy positioning note.

### BACKGROUND OF THE INVENTION

A radiation therapy positioning note is a positioning note which is noted, prior to a radiation therapy, on a patient's predetermined treating position in order to thereafter accurately perform a radiation therapy treatment on the predetermined treating position during radiation therapy operations.

In prior operation, a medical staff uses a permanent marker pen to draw a note line as the radiation therapy positioning note on the patient's predetermined treating position and thereafter pastes a waterproof tape on the note line.

However, the note line drawn by the permanent marker pen causes the inconvenience for the patent when the patent takes a bath to wipe and wash body where the note line is positioned. Furthermore, the note line drawn by the permanent marker pen is easily faded and the ink of the permanent marker pen may penetrate into the human skin to cause damage to the skin. Therefore, there is a need for improvement.

### SUMMARY OF THE INVENTION

Therefore, one objective of the present invention is to provide a marking method for marking a radiation therapy positioning note, which can set the radiation therapy positioning note on a radiation therapy target object but not causes inconvenience to patents' daily life and not damages the patents' skin.

In order to overcome the technical problems in prior art, the present invention provides a marking method for marking a radiation therapy positioning note, comprising in sequence: a preparing step of providing a radiation therapy positioning sticker, wherein the radiation therapy positioning sticker includes a pattern layer and a release layer disposed on an outer surface of the pattern layer, and the radiation therapy positioning note is formed on the outer surface of the pattern layer; a transferring step of attaching the radiation therapy positioning sticker onto a predetermined note position of a radiation therapy target object in a manner that an inner surface of the pattern layer faces forward the predetermined note position and covers the predetermined note position; and a removing step of peeling off the release layer from the outer surface of the pattern layer to outwardly expose the radiation therapy positioning note, which is formed on the outer surface of the pattern layer covering the predetermined note position.

In one embodiment of the present invention, the marking method is provided, further comprising, prior to the transferring step, a pointing step of forming a temporary pointing note on the predetermined note position in a non-contact and non-destructive manner, and during the transferring step, the radiation therapy positioning sticker being attached onto the predetermined note position according to the temporary pointing note.

In one embodiment of the present invention, the marking method is provided, wherein in the pointing step, the temporary pointing note is a light pointing note which is formed by projecting a laser beam onto the predetermined note position.

In one embodiment of the present invention, the marking method is provided, wherein in the transferring step, the radiation therapy positioning sticker is, in a press-transfer printing manner, attached onto the predetermined note position of the radiation therapy target object.

In one embodiment of the present invention, the marking method is provided, wherein in the transferring step, the radiation therapy positioning sticker is, in a water-transfer printing manner, attached onto the predetermined note position of the radiation therapy target object.

In one embodiment of the present invention, the marking method is provided, wherein the pattern layer of the radiation therapy positioning sticker is a thin-film polymer material layer.

In one embodiment of the present invention, the marking method is provided, wherein an adhesive layer is provided respectively on the outer surface and the inner surface of the pattern layer of the radiation therapy positioning sticker.

In one embodiment of the present invention, the marking method is provided, wherein a surface area of the pattern layer is smaller than that of the release layer.

With the technical means adopted by the present invention, the radiation therapy positioning note is attached onto the predetermined note position of the radiation therapy target object, i.e., the patent, in a transfer printing manner such as a press-transfer printing manner or a water-transfer printing manner, which is with merit that the note would not be easily peeled off from the predetermined note position. The above merit also brings daily life convenience for a patent. In addition, the marking method of the present invention ensures the skin health without hurting the skin. Moreover, the pointing step also improves the positional accuracy by attaching the radiation therapy positioning sticker onto the predetermined note position. Preferably, the pointing step of the present invention applies a non-contact and non-destructive manner by projecting the laser beam on the predetermined note position to form a temporary pointing note.

### BRIEF DESCRIPTION OF THE DRAWINGS

FIG. 1 is a schematic flowchart illustrating a marking method for marking a radiation therapy positioning note according to a first embodiment of the present invention;

FIG. 2 is a schematic drawing illustrating a preparing step of the marking method according to the first embodiment of the present invention;

FIG. 3 is a schematic drawing illustrating a transferring step of the marking method according to the first embodiment of the present invention;

FIG. 4 is a schematic drawing illustrating a removing step of the marking method according to the first embodiment of the present invention;

FIG. 5 is a schematic flowchart illustrating a marking method according to a second embodiment of the present invention; and

FIG. 6 is a schematic drawing illustrating a pointing step of the marking method according to the second embodiment of the present invention.

### DETAILED DESCRIPTION OF THE PREFERRED EMBODIMENTS

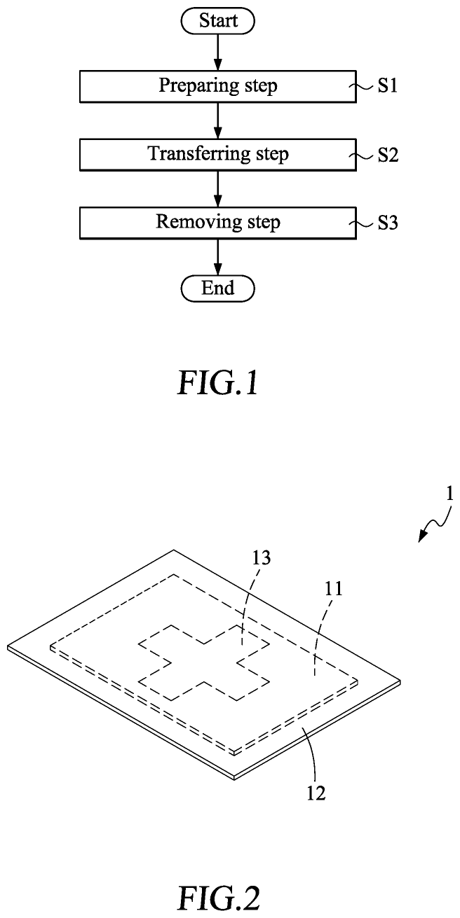

The preferred embodiments of the present invention are described in detail below with reference to FIG. 1 to FIG. 6. The description is used for explaining the embodiments of the present invention only, but not for limiting the scope of the claims.

As shown in FIG. 1 to FIG. 4, a marking method for marking a radiation therapy positioning note according to an embodiment of the present invention comprises in sequence: a preparing step S1, a transferring step S2, and a removing step S3.

The preparing step S1: provide a radiation therapy positioning sticker 1, wherein the radiation therapy positioning sticker 1 includes a pattern layer 11 and a release layer 12 disposed on an outer surface of the pattern layer 11, and the radiation therapy positioning note 13 is formed on the outer surface of the pattern layer 11.

The transferring step S2: attach the radiation therapy positioning sticker 1 onto a predetermined note position of a radiation therapy target object P in a manner that an inner surface of the pattern layer 11 faces forward the predetermined note position and covers the predetermined note position.

The removing step S3: peel off the release layer 12 from the outer surface of the pattern layer 11 to outwardly expose the radiation therapy positioning note 13, which is formed on the outer surface of the pattern layer 11 covering the predetermined note position.

Please refer to FIG. 1 and FIG. 2, according to the marking method of the embodiment of the present invention, in the preparing step S1, the pattern layer 11 of the radiation therapy positioning sticker 1 is a water-resistant thin-film polymer material layer, i.e., the pattern layer 11 will not easily come off or decompose when exposed to water. However, the present invention is not limited to this, and the pattern layer 11 of the radiation therapy positioning sticker 1 may be a thin-film polymer material layer which is not water-resistant. Moreover, an adhesive layer is provided respectively on the outer surface and the inner surface of the pattern layer 11, wherein the adhesive layer provided on the inner surface of the pattern layer 11 enables the pattern layer 11 to be attached onto the predetermined note position, and the adhesive layer provided on the outer surface of the pattern layer 11 enables the release layer 12 to be disposed on the outer surface of the pattern layer 11.

As shown in FIG. 2, according to the marking method of the embodiment of the present invention, the release layer 12 is a transparent release paper which can be easily removed from the outer surface of the pattern layer 11. However, the release layer 12 may be a general release paper having a substrate made of paper, cloth, plastic film, etc. and coated, on one or both surfaces of the substrate, with a release treatment agent with organic or inorganic release properties, such as silicone resin.

As shown in FIG. 2, a surface area of the pattern layer 11 is smaller than that of the release layer 12. However, the surface area of the pattern layer 11 may also be equal to or larger than that of the release layer 12. The radiation therapy positioning note 13 is formed on the outer surface of the pattern layer 11 of the radiation therapy positioning sticker 1. In the embodiment, the radiation therapy positioning note 13 is a crisscross pattern and is formed on a central portion of the outer surface of the pattern layer 11. However, the present invention is not limited to this. The radiation therapy positioning note 13 may be a pattern of any shape or numerical symbol, and the radiation therapy positioning note 13 may be formed on any portion of the outer surface of the pattern layer 11 or may completely cover the outer surface of the pattern layer 11. In addition, the radiation therapy positioning note 13 may also be the pattern layer 11.

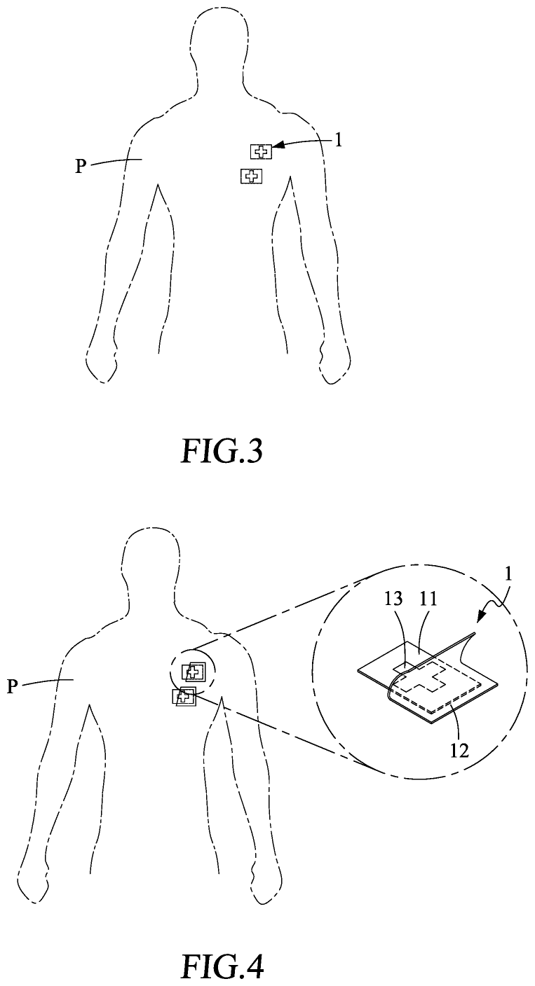

Please refer to FIG. 1 and FIG. 3, according to the marking method of the embodiment of the present invention, in the transferring step S2, the pattern layer 11 of the radiation therapy positioning sticker 1 is, in a press-transfer printing manner, transferred to the predetermined note position of the radiation therapy target object P. The press-transfer printing is a technology that transfers a shape or line pattern to a surface of a different material object by using an effect produced by an applying force. Specifically, the transferring step S2 is performed by: putting the inner surface of the pattern layer 11 to face forward the predetermined note position; making sure that the inner surface of the pattern layer 11 covers the predetermined note position; and uniformly applying a pressing force to the radiation therapy positioning sticker 1 to attach the pattern layer 11 to the predetermined note position so that the pattern layer 11 is attached onto the predetermined note position through the adhesive layer provided on the inner surface of the pattern layer 11.

Please refer to FIG. 1 and FIG. 4, according to the marking method of the embodiment of the present invention, in the removing step S3, the release layer 12 is directly removed from the outer surface of the pattern layer. Specifically, since the release layer 12 is the release paper, the release layer can be easily peeled off from the outer surface of the pattern layer. After the release layer 12 is removed, the pattern layer 11 will be attached to the predetermined note position and the radiation therapy positioning note 13 formed on the outer surface of the pattern layer 11 will expose outwardly, so that the portion of the radiation therapy target object where the radiation therapy treatment is to be performed can be clearly identified during subsequent radiation therapy operations.

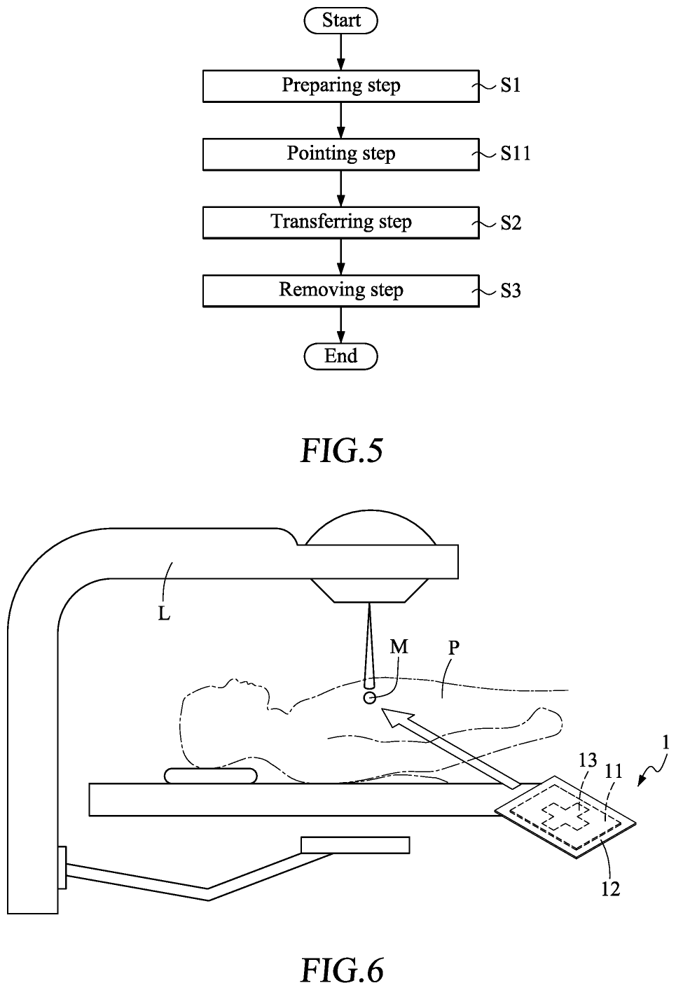

Please refer to FIG. 5 and FIG. 6, the difference between a marking method according to a second embodiment of the present invention and that of the first embodiment of the present invention is that: the marking method further comprises, prior to the transferring step S2, a pointing step S11 of forming a temporary pointing note M on the predetermined note position in a non-contact and non-destructive manner.

As shown in FIG. 5 and FIG. 6, according to the marking method of the second embodiment of the present invention, specifically in the pointing step S11, the non-contact and non-destructive manner which is used is a manner of pointing by projecting a laser beam, that is, the temporary pointing note M is a light pointing note which is formed by projecting a laser beam onto the predetermined note position. The manner of pointing by projecting the laser beam is to project a light emitted from laser equipment L onto the predetermined note position of the radiation therapy target object P. The manner of pointing by projecting the laser beam is with merit that it can accurately point the predetermined note position and does not damage the radiation therapy target object P. However, the present invention is not limited to this, and the pointing step S11 may use other manner to form the temporary pointing note M on the predetermined note position, for example, a permanent marker pen may be used to draw a crisscross pattern or the likes on the predetermined note position.

Moreover, according to the marking method of the second embodiment of the present invention, the pointing step S11 can be performed prior to the preparing step S1. In other words, prior to the preparing step S1, the temporary pointing note M is formed on the predetermined note position by projecting a laser beam or drawing with a permanent marker pen, and after that the preparing step S1, the transferring step S2, and the removing step S3 are performed in sequence.

In other embodiment of the present invention, in the transferring step, the radiation therapy positioning sticker may be transferred to the predetermined note position of the radiation therapy target object P in other transfer printing manner, such as a water-transfer printing manner. The water-transfer printing is a technology that transfers a shape or line pattern to a surface of a different material object by using an effect of water.

Specifically, the difference between the marking method using the water-transfer printing according to the embodiment of the present invention and the marking method of the first embodiment of the present invention is that: in the preparing step, the radiation therapy positioning sticker is a water-transfer printing sticker of which an adhesive layer provided on the inner surface of the pattern layer of the radiation therapy positioning sticker is made of a water-insoluble polymer mucosal material enabling the inner surface of the pattern layer to be attached onto the predetermined note position. The transferring step is performed by: dipping or soaking the radiation therapy positioning sticker in water, e.g., directly dipping or soaking the radiation therapy positioning sticker in a container filled with water; putting the inner surface of the pattern layer of the radiation therapy positioning sticker dipped or soaked in water to face forward the predetermined note position of the radiation therapy target object P; making sure that the inner surface of the pattern layer covers the predetermined note position; and directly attaching the radiation therapy positioning sticker to the predetermined note position. Finally, the release layer is peeled off so that the pattern layer is attached onto the predetermined note position through the adhesive layer provided on the inner surface of the pattern layer.

With the structure mentioned above, the marking method of the present invention can accurately transfer the radiation therapy positioning note 13 to the predetermined note position of the radiation therapy target object P in order to perform, by identifying the radiation therapy positioning note 13, the radiation therapy treatment on the position of the radiation therapy target object P where the radiation therapy treatment is to be performed in subsequent radiation therapy operations. In the marking method of the present invention, the pattern layer 11 of the radiation therapy positioning sticker 1 is attached onto the predetermined note position of the radiation therapy target object P, i.e., the patent, in the transfer printing manner such as the press-transfer printing manner or the water-transfer printing manner, which is with merit the marking method is easy to implement, and the pattern layer 11 of the radiation therapy positioning sticker 1 ensures the skin health without hurting the skin of the radiation therapy target object P. Moreover, the radiation therapy positioning sticker 1 is effective for a long time. Even if the radiation therapy positioning sticker 1 is touched by the radiation therapy target object P who is sweating or is taking a bath, the pattern layer 11 of the radiation therapy positioning sticker 1 will not be easily peeled off from the predetermined note position of the radiation therapy target object P. Furthermore, the radiation therapy positioning sticker 1 may be provided in the form of a roll, such as a tape roll, so that the radiation therapy positioning sticker 1 can be pulled off from the radiation therapy positioning sticker roll and be cut to a certain length for use. In addition, the pointing step S11 also improves the positional accuracy by attaching the radiation therapy positioning sticker 1 onto the predetermined note position in the transferring step S2.

The above description should be considered as only the discussion of the preferred embodiments of the present invention. However, a person having ordinary skill in the art may make various modifications without deviating from the present invention. Those modifications still fall within the scope of the present invention.

What is claimed is:

1. A marking method for marking a radiation therapy positioning note, comprising in sequence:
a preparing step of providing a radiation therapy positioning sticker, wherein the radiation therapy positioning sticker includes a pattern layer and a release layer disposed on an outer surface of the pattern layer, and the radiation therapy positioning note is formed on the outer surface of the pattern layer;
a transferring step of attaching the radiation therapy positioning sticker onto a predetermined note position of a radiation therapy target object in a manner that an inner surface of the pattern layer faces forward the predetermined note position and covers the predetermined note position; and
a removing step of peeling off the release layer from the outer surface of the pattern layer to outwardly expose the radiation therapy positioning note, which is formed on the outer surface of the pattern layer covering the predetermined note position.

2. The marking method as claimed in claim 1, further comprising, prior to the transferring step, a pointing step of forming a temporary pointing note on the predetermined note position in a non-contact and non-destructive manner, and
during the transferring step, the radiation therapy positioning sticker being attached onto the predetermined note position according to the temporary pointing note.

3. The marking method as claimed in claim 2, wherein in the pointing step, the temporary pointing note is a light pointing note which is formed by projecting a laser beam onto the predetermined note position.

4. The marking method as claimed in claim 1, wherein in the transferring step, the radiation therapy positioning sticker is, in a press-transfer printing manner, attached onto the predetermined note position of the radiation therapy target object.

5. The marking method as claimed in claim 1, wherein in the transferring step, the radiation therapy positioning sticker is, in a water-transfer printing manner, attached onto the predetermined note position of the radiation therapy target object.

6. The marking method as claimed in claim 1, wherein the pattern layer of the radiation therapy positioning sticker is a thin-film polymer material layer.

7. The marking method as claimed in claim 1, wherein an adhesive layer is provided respectively on the outer surface and the inner surface of the pattern layer of the radiation therapy positioning sticker.

8. The marking method as claimed in claim 1, wherein a surface area of the pattern layer is smaller than that of the release layer.
